(19) United States
(12) Patent Application Publication
(10) Pub. No.: US 2013/0129632 A1
(43) Pub. Date: May 23, 2013

(54) Quantum dot materials, methods for making them, and uses thereof

(71) Applicants: Kam W. Leong, Durham, NC (US); Chai-Hoon Quek, Durham, NC (US)

(72) Inventors: Kam W. Leong, Durham, NC (US); Chai-Hoon Quek, Durham, NC (US)

(21) Appl. No.: 13/573,690

(22) Filed: Oct. 3, 2012

Related U.S. Application Data
(60) Provisional application No. 61/542,603, filed on Oct. 3, 2011.

(51) Int. Cl. A61K 49/00; A61K 49/04; A61K 49/08

(52) U.S. Cl. CPC A61K 49/00; A61K 49/085; A61K 49/04; Y10S 977/774; Y10S 977/927; Y10S 977/928; Y10S 977/93; B82Y 5/00
USPC 424/9.32; 424/9.6; 424/9.42; 977/774; 977/927; 977/928; 977/930

(57) ABSTRACT

The disclosure provides quantum dot materials, compositions and methods useful in the treatment of various disorders. In particular, the disclosure provides cadmium-free and lead-free quantum dots.

### CROSS-REFERENCE TO RELATED APPLICATIONS

This application claims the benefit of the filing date of U.S. Provisional Patent Application Ser. No. 61/542,603, filed Oct. 3, 2011, which is hereby incorporated by reference in its entirety.

### BACKGROUND OF THE INVENTION

1. Field of the Invention

This disclosure relates to quantum dot materials, compositions and methods useful in the diagnosis and treatment of various disorders. In particular, the disclosure provides cadmium-free and lead-free quantum dots.

2. Description of Related Art

Quantum dot materials are emerging as an important class of nanomaterials for application in many commercial consumer and clinical products. The diverse applications of quantum dots are attributed to their superior optical properties, which include narrow, symmetric and size/composition tunable emission spectra, high photostability, and broad range excitation that renders possible the simultaneous detection of multiple targets. Quantum dots offer attractive characteristics as a new class of fluorescent probes for molecular, cellular and in vivo imaging. Quantum dots are comprised of binary combinations of elements from periodic groups of II-VI (CdSe and CdTe), III-V (InP and InAs) or IV-VI (PbS) and afford a range of emission wavelengths from the UV to the near infrared. To date, the most widely studied quantum dots that are readily available from commercial sources are formed from CdSe, CdTe and PbSe. While these traditional cadmium- and lead-containing quantum dots have been widely used in biomedical research, diagnostics, and drug delivery, the cytotoxicity arising from the release of Cd²⁺ and Pb²⁺ ions caused by the degradation of the surface coating is deemed to be a shortfall of these quantum dots for long-term cellular and in vivo imaging. With the implementation of the “Restriction of Hazardous Substances Directive” (RoHS) in February 2003 by the European Union, there is an increasing desire to phase out the use of cadmium, lead and other heavy metals containing quantum dots in consumer products.

Quantum dots are typically synthesized using organometallic synthesis techniques. These techniques typically employ pyrophoric precursors, stabilized with hydrophobic ligands, organic solvents, and high temperatures.

### SUMMARY OF THE INVENTION

In a broad aspect, the disclosure encompasses quantum dot materials, conjugates comprising the quantum dot materials, diagnostic compositions containing the quantum dot materials or conjugates, and methods employing such quantum dot materials and conjugates in methods useful in the treatment of diseases and/or disorders.

Thus, one aspect of the disclosure provides a quantum dot material comprising a plurality of particles, each particle comprising:
a core having the formula Zn₁₋ₓAgₓY in which Y is S, Se or Te, and x is 0.001-0.05; and
at least a partial layer of stabilizing ligand substantially surrounding the core, the stabilizing ligand being glutathione, thioglycolic acid, 3-mercaptopropionic acid, mercaptoethanol, 3-mercaptopropane-1,2-diol, thiolactic acid, cysteine, cysteamine, or a combination thereof;
   wherein the particles have an average diameter in the range of about 2 nm to about 50 nm.

The disclosure also provides diagnostic compositions comprising a quantum dot material of the disclosure and at least one diagnostically acceptable carrier, solvent, adjuvant or diluent.

The disclosure provides simple and direct methods of preparing a quantum dot material of the disclosure and the intermediates used in those methods.

The disclosure also provides a conjugate comprising a quantum dot material of the disclosure and a nanoparticle.

The disclosure further provides a conjugate comprising a quantum dot material of the disclosure and a targeting moiety.

The disclosure further provides methods for monitoring delivery of a targeting moiety to an organism or biomaterial using the conjugates of the disclosure.

The disclosure also provides methods for monitoring delivery of a targeting moiety to a subject using the conjugates of the disclosure.

### DESCRIPTION OF DRAWINGS

FIG. 1. Fluorescence intensity spectra observed at different time. (a) reaction at pH 5, (b) reaction at pH 6, and (c) reaction at pH 7. Fluorescence intensity spectra were recorded with excitation at 488 nm. The data is shown in sequential order where the peak with the lowest fluorescence intensity (in arbitrary units, “A.U.”) represents the earliest time point (e.g., 5 minutes) and the peak with the highest fluorescence intensity represents the latest time point. For example, in (a) the peak with the highest intensity represents 100 minutes, the peak with the second highest intensity represents 70 minutes, the peak with the third highest intensity represents 50 minutes, etc. In (b) and (c), in addition to increasing fluorescence intensity, the wavelength (in nm) also increases sequentially, over time.

FIG. 2. Effect of concentration of Zn²⁺ on rate of reaction. (a) 3 mmol ZnAc₂, (b) 5 mmol ZnAc₂, and (c) 7.5 mmol ZnAc₂. Fluorescence intensity spectra were recorded with excitation at 488 nm. The data is shown in sequential order where the peak with the lowest fluorescence intensity (in A.U.) represents the earliest, time point (e.g., 10 minutes) and the peak with the highest fluorescence intensity represents the latest time point. For example, in (a) the peak with the highest intensity represents 75 minutes, the peak with the second highest intensity represents 60 minutes, the peak with the third highest intensity represents 45 minutes, etc.

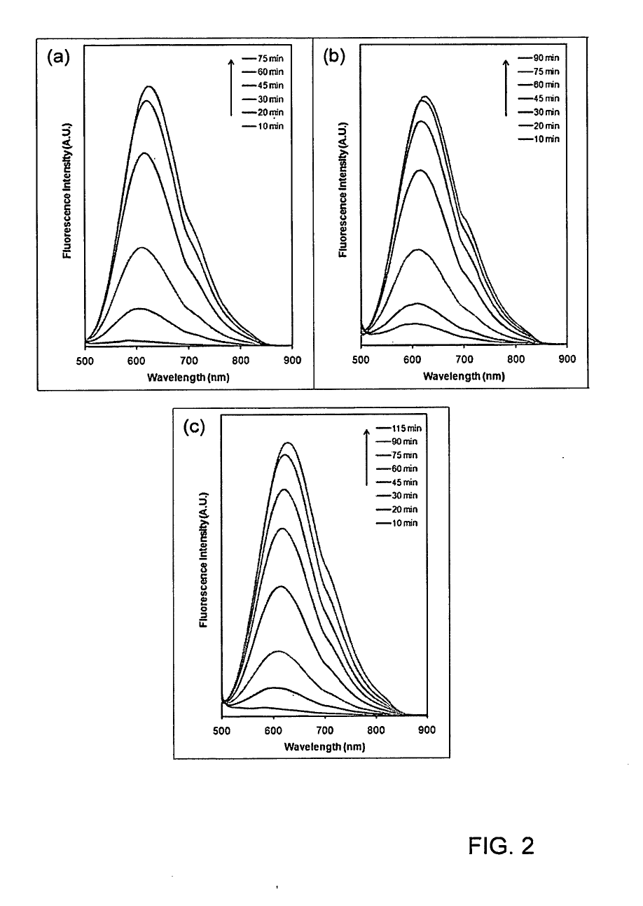

FIG. 3. Effect of concentration of Ag⁺ on rate of reaction. (a) 0.05 mmol AgAc (b) 0.10 mmol AgAc, and (c) 0.15 mmol AgAc. The data is shown in sequential order where the peak with the lowest fluorescence intensity (in A.U.) represents the earliest time point (e.g., 5 minutes) and the peak with the highest fluorescence intensity represents the latest time point. For example, in (a) the peak with the highest intensity represents 95 minutes, the peak with the second highest intensity represents 70 minutes, the peak with the third highest intensity represents 50 minutes, etc.

FIG. 4. Effect of concentration of Se²⁻ on rate of reaction. (a) 0.4 mmol of NaHSe, (b) 0.6 mmol of NaHSe, and (c) 0.8 mol of NaHSe. The data is shown in sequential order where the peak with the lowest fluorescence intensity (in A.U.) represents the earliest time point (e.g., 10 minutes) and the peak with the highest fluorescence intensity represents the latest time point. For example, in (a) the peak with the highest intensity represents 80 minutes, the peak with the second highest intensity represents 65 minutes, the peak with the third highest intensity represents 50 minutes, etc.

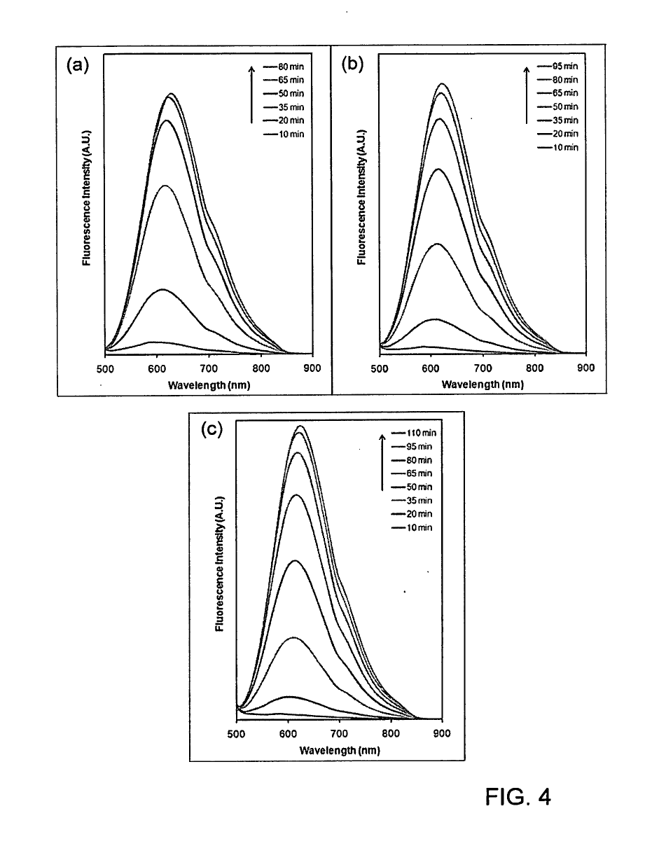

FIG. 5. Effect of GSH at pH 7 on rate of reaction. (a) 6 mmol of GSH, (b) 10 mmol of GSH, and (c) 13 mol of GSH. The data is shown in sequential order where the peak with the lowest wavelength (in nm) represents the earliest time point (e.g., 5 minutes) and the peak with the highest wavelength represents the latest time point. For example, in (a) the peak with the highest wavelength represents 175 minutes, the peak with the second highest wavelength represents 110 minutes, the peak with the third highest wavelength represents 95 minutes, etc.

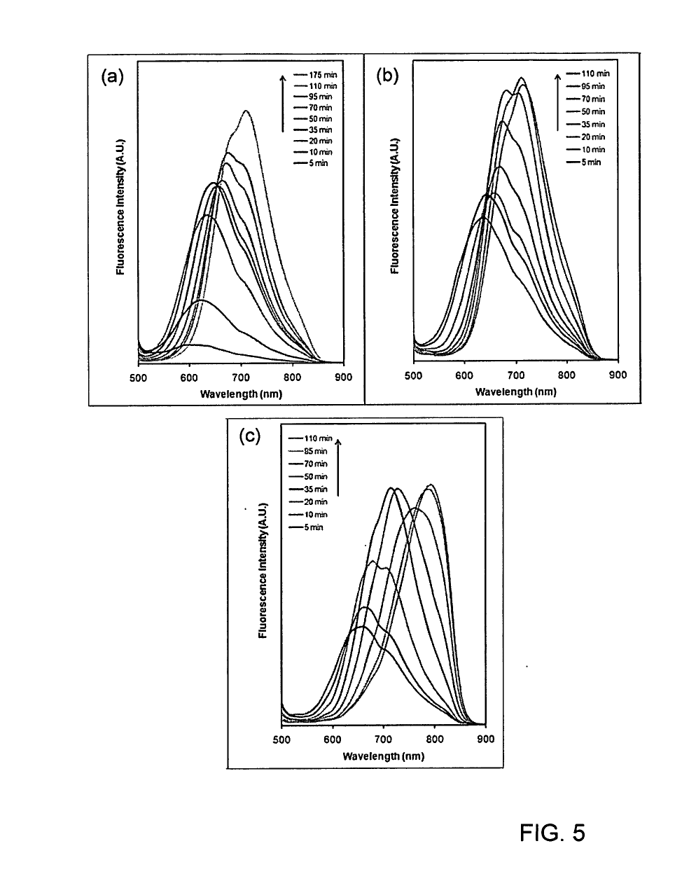

FIG. 6. Absorption and fluorescence emission spectra of different sizes of the quantum dots of the disclosure. Fluorescence spectra were recorded with excitation at 420 nm. (a) QD710, (b) QD620, and (c) QD510.

FIG. 7. (a) Photograph of the quantum dots of the disclosure under illumination with 365 nm UV light; QD510 is cyan, QD620 is yellow, and QD710 is red. (b) Transmission electron microscopy (TEM) image of QD710 with an average diameter of about 10 nm.

FIG. 8. Powder X-ray diffraction pattern of QD710 revealed a zinc blende cubic crystal structure.

FIG. 9. In vitro cytotoxicity of the quantum dots of the disclosure. (a) 24 h and (b) 48 h treatment on macrophage cell line (RAW 264.7 cells). (c) 24 h and (d) 48 h treatment on human mesenchymal stem cells. Poly(ethyleneimine) (PEI) was used as the positive control. ZnSeAg720 is QD710; ZnSeAg680 is QD680; and ZnSeAg645 is QD645. Individual bars at each concentration, from left to right, represent ZnSeAg720, ZnSeAg680, ZnSeAg645, and PEI.

FIG. 10. Bright-field and fluorescence images of (a,b) RAW264.7 cells and (c,d) hMSC incubated with QD710 for 24 h. Emissions wavelength: 610 nm.

FIG. 11. In vivo imaging of QD710 and commercially available quantum dots (Invitrogen) (QD800-NH₂) after footpad injections in Nu/Nu mice. The fluorescence pattern was analyzed at different time interval post-injection, (a) 5 hr, (b) 1 day, (c) 3 days, and (d) 7 days. The organs imaged are (left-to-right) kidneys, liver, spleen, intestines, popiteal lymph nodes, and stomach. Localized fluorescence is indicated with arrows.

FIG. 12. Histology of stomach sections 7 days post injection with QD710: (a) mouse injected with 50 μL PBS, (b) with 50 μL QD710 (50 mg/mL) and (c) with 50 μL of QD800-NH₂ (Qdot 800 ITK amino (PEG) quantum dots, Invitrogen, 0.16 μM). Magnification is 20 times and white scale corresponds to 50 μm.

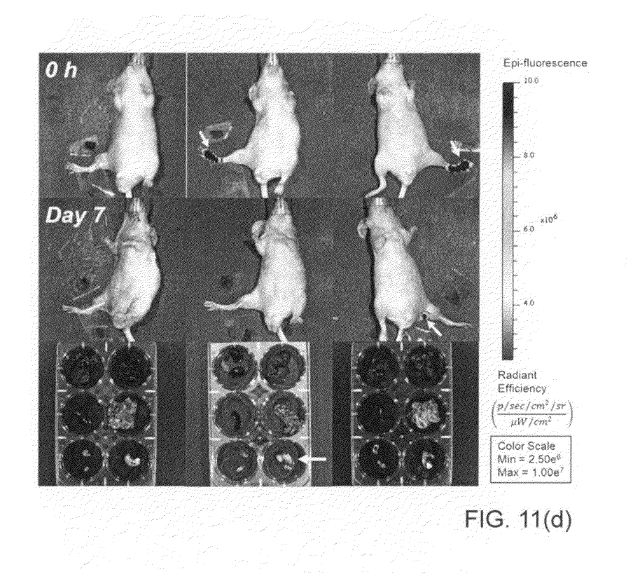

FIG. 13. Histology of popiteal lymph node sections 7 days post injection with QD710: (a) mouse injected with 50 μL PBS, (b) with 50 μL QD710 (50 mg/mL) and (c) with 50 μL of QD800-NH₂ (Qdot 800 ITK amino (PEG) quantum dots, Invitrogen, 0.16 μM). Magnification is 20 times and white scale corresponds to 50 μm.

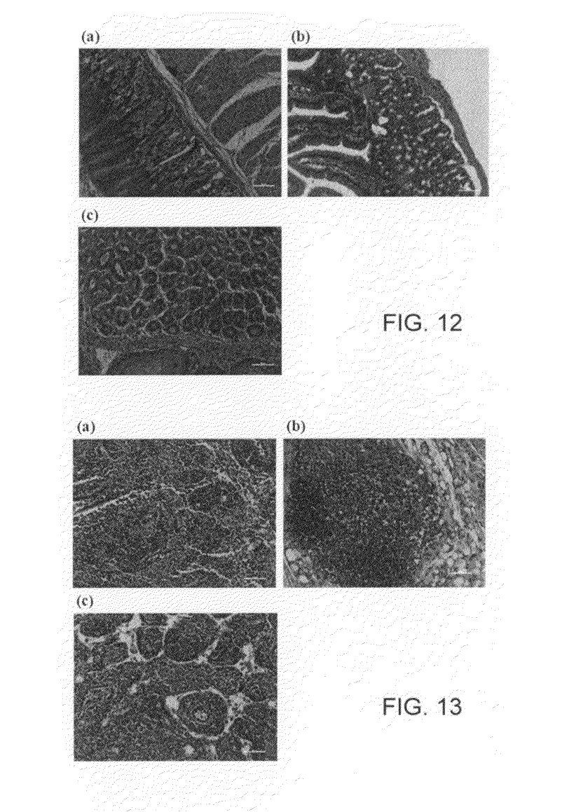

FIG. 14. (a) TEM of chitosan/heparin nanocomplexes (NCs) with an average diameter of about 60-120 nm. Fluorescence imaging of mice injected with PBS (left panels) and QD-labeled heparin/chitosan nanocomplexes (right panels) at (b) 0 h, and (c) 4 h post-injection. Localized fluorescence is indicated with arrows.

### DETAILED DESCRIPTION OF THE INVENTION

In one embodiment, the disclosure provides a quantum dot material comprising a plurality of particles, each particle comprising:
a core having the formula Zn₁₋ₓAgₓY in which Y is S, Se or Te, and x is 0.001-0.05; and
at least a partial layer of stabilizing ligand substantially surrounding the core, the stabilizing ligand being glutathione, thioglycolic acid, 3-mercaptopropionic acid, mercaptoethanol, 3-mercaptopropane-1,2-diol, thiolactic acid, cysteine, cysteamine, or a combination thereof;
   wherein the particles have an average diameter in the range of about 2 nm to about 50 nm.

In another embodiment, the disclosure provides a quantum dot material as described above, wherein Y is Se or Te.

In yet another embodiment, the disclosure provides a quantum dot material as described above, wherein Y is Se. This quantum dot material has a core of formula Zn₁₋ₓAgₓSe.

In yet another embodiment, the disclosure provides a quantum dot material as described above, wherein Y is Te. This quantum dot material has a core of formula Zn₁₋ₓAgₓTe.

In one embodiment, the disclosure provides a quantum dot material as described above, wherein x is 0.001-0.03. In another embodiment, x is 0.005-0.035. In yet another embodiment, x is 0.01-0.03. The disclosure further provides quantum dot material as described wherein x is about 0.01. The disclosure also provides quantum dot material as described wherein x is about 0.02. The disclosure further provides quantum dot material as described wherein x is about 0.03.

In another embodiment, the disclosure provides a quantum dot material as described above, wherein the stabilizing ligand is glutathione, thioglycolic acid, or 3-mercaptopropionic acid. In another embodiment, the stabilizing ligand is glutathione.

In one embodiment, the disclosure provides a quantum dot material as described above, wherein the material has a fluorescence emission at a wavelength in the visible or near-infrared. The fluorescence emission wavelength is about 400 nm to about 1100 nm. In one embodiment, the fluorescence emission wavelength is about 620 nm to about 900 nm. In another embodiment, the fluorescence emission wavelength is about 620 nm to about 800 nm.

In another embodiment, the disclosure provides a quantum dot material as described above, wherein the particles have an average diameter in the range of about 2 to about 15 nm. In another embodiment, the particles have an average diameter in the range of about 2 to about 10 nm.

In one embodiment, the disclosure provides method of preparing a quantum dot material as described above, the method comprising:

(a) mixing a Zn precursor, a Ag precursor, and the stabilizing ligand in a solvent;

(b) adding a solution containing a Y precursor to the mixture of (a);

(c) allowing the plurality of particles to form; and

(d) precipitating the formed quantum dot.

The zinc precursor can be, for example, a Zn(II) salt, preferably water soluble. For example, in certain embodiments, the disclosure provides a method as described above, wherein Zn precursor is zinc acetate or zinc nitrate. In other embodiments, the disclosure provides a method as described above, wherein Zn precursor is zinc acetate. In other embodiments, the disclosure provides a method as described above, wherein Zn precursor is zinc nitrate.

The silver precursor can be, for example, a Ag(II) salt, preferably water soluble. In some embodiments, the disclosure provides a method as described above, wherein Ag precursor is silver acetate or silver nitrate. In other embodiments, the disclosure provides a method as described above, wherein Ag precursor is silver acetate. In other embodiments, the disclosure provides a method as described above, wherein Ag precursor is silver nitrate.

In one embodiment, the disclosure provides a method as described above, wherein the stabilizing ligand is glutathione, thioglycolic acid, 3-mercaptopropionic acid, thioglycolic acid, mercaptoethanol, 3-mercaptopropane-1,2-diol, thiolactic acid, cysteine, or cysteamine. In another embodiment, the stabilizing ligand is glutathione, thioglycolic acid, or 3-mercaptopropionic acid. In yet another embodiment, the stabilizing ligand is glutathione.

In one embodiment, the disclosure provides a method as described above, wherein the Y precursor is NaHSe.

In another embodiment, the disclosure provides a method as described above, wherein the molar ratio of Zn precursor to Ag precursor is between 30:1 and 100:1. In one embodiment, the molar ratio of Zn precursor to Ag precursor is between about 50:1 and 100:1.

In certain embodiment, the disclosure provides a method as described above, wherein the molar ratio of Zn precursor to Y precursor is between 10:1 and 5:1. In another embodiment, the molar ratio of Zn precursor to Y precursor is about 8:1.

In one embodiment, the disclosure provides a method as described above, wherein the molar ratio of Zn precursor to the stabilizing ligand is between 1:1 and 1:4. In another embodiment, the molar ratio of Zn precursor to the stabilizing ligand is between 1:1.5 and 1:3. In yet another embodiment, the molar ratio of Zn precursor to the stabilizing ligand is about 1:2.25.

In some embodiments, the disclosure provides a method as described above, wherein the solvent is water, a water miscible solvent, or a mixture thereof. In one embodiment, the solvent is water. In another embodiment, the solvent is a water miscible solvent, either as single component or as a mixture with water. Examples of suitable water miscible solvents include ethanol, isopropanol, isopropyl myristate, glycerin, propylene glycol, polyethylene glycol, 2-(2-ethoxyethoxy)ethanol (Transcutol®) and the like. The person of skill in the art will understand that other water miscible solvents can be used in practicing certain aspects of the methods disclosed herein.

In another embodiment, the solvent is a mixture of water and a water miscible solvent. In certain methods, the water in the mixture of water and a water miscible solvent can be present in an amount of about 50 to about 99.9 volume percent, about 80 to about 99 volume percent, about 90 to about 99 volume percent, or about 95 to about 99 volume percent, based on the total volume of the mixture. In some embodiments, the water component can be present in an amount of more than about 95 volume percent.

In certain embodiments, the disclosure provides a method as described above performed at a temperature of about 0° C. to about 50° C., about 10° C. to about 40° C., about 15° C. to about 35° C., about 20° C. to about 30° C., about 21° C. to about 27° C., about 20° C. to about 25° C., or about 25° C. In another embodiment, the method as described above is performed at room temperature.

In one embodiment, the disclosure provides a method as described above, wherein the pH of the solution is from about 10 to about 12. In another embodiment, the disclosure provides a method as described above, wherein the pH is about 5 to about 8. In yet another embodiment, the pH is about 5 to about 7. In yet another embodiment, the pH is about 5. In another embodiment, the pH is about 6. In yet another embodiment, the pH is about 7.

In one embodiment, the disclosure provides a method as described above, wherein the time between performing step (b) and step (d) is 15 min to 120 min.

### Applications

One aspect of the disclosure provides a conjugate comprising a quantum dot material as described above and a nanoparticle.

In one embodiment, the disclosure provides a conjugate wherein the nanoparticle is heparin/chitosan. In particular, heparin is conjugated to the quantum dot material, and the nanoparticle is formed with chitosan. The conjugate comprising the quantum dot material and heparin/chitosan nanoparticle is soluble in water.

Another aspect of the disclosure provides a conjugate comprising a quantum dot material as described above and a targeting moiety.

In one embodiment, the targeting moiety is a protein, a fragment of a protein, or a nucleic acid. In another embodiment, protein or fragment thereof for use as a targeting moiety is an antigen, an epitope of an antigen, an antibody, or an antigenically reactive fragment of an antibody. In another embodiment, protein is streptavidin. In another embodiment, nucleic acid is a single-stranded oligonucleotide comprising a stem and loop structure and the hydrophilic attachment group is attached to one end of the single-stranded oligonucleotide and a quenching moiety is attached to the other end of the single-stranded oligonucleotide and the quenching moiety quenches the luminescent semiconductor quantum dot. The targeting moiety should not render the quantum dot water-insoluble.

An additional aspect of the disclosure provides a method for monitoring delivery of a targeting moiety to an organism or biomaterial, the method comprising contacting the organism or biomaterial with a conjugate as described above, and analyzing the cell to detect one or more of the presence, absence, amount and location of the conjugate in the organism or biomaterial.

An additional aspect of the disclosure provides a method for monitoring delivery of a targeting moiety to a subject comprising administering to a subject an effective amount of a conjugate as described above, and detecting the presence, absence, or amount of the conjugate.

In one embodiment, the disclosure provides a method for monitoring delivery of a targeting moiety wherein the detection is magnetic resonance imaging (MRI), X-ray imaging, computed tomography (CT), or electron spin resonance (ESR) imaging.

In another embodiment, the disclosure provides a method for monitoring delivery of a targeting moiety wherein the detection is performed without the further addition of a contrast reagent.

In one embodiment, the disclosure provides a method for optical imaging comprising administering to a subject an effective amount of a conjugate as described above. In another embodiment, the disclosure provides a method for labeling cells (fixed and live) and tissues, long-term cell trafficking, multicolor cell imaging, fluorescence resonance energy transfer (FRET)-based sensing, or sentinel lymph-node mapping, comprising administering to a subject an effective amount of a conjugate as described above.

The quantum dot(s) of the disclosure have broad application for the real-time observation of cellular mechanisms in living cells, e.g., ligand-receptor interaction and molecular trafficking, due to the increased photostability of the quantum dot(s). Thus, the quantum dot(s) of the invention can be used in various diagnostic assays, including, but not limited to, the detection of viral infection, cancer, cardiac disease, liver disease, genetic diseases, and immunological diseases. The quantum dot(s) of the disclosure can used in a diagnostic assay to detect certain viruses, by, for example, (a) removing a sample to be tested from a patient; (b) contacting the sample with a luminescent conjugate comprising a quantum dot material and a targeting moiety as described above, wherein the targeting moiety is an antibody or antigenically reactive fragment thereof that binds to the virus; and (c) detecting the luminescence, wherein the detection of luminescence indicates that the virus is present in the sample. The patient sample can be a bodily fluid, such as saliva, tears, blood, serum, or urine.

The quantum dot(s) of the disclosure can be used in a diagnostic assay to determine ultra-low-level viral loads of certain viruses by detecting the viral nucleic acid. Determining the viral load of a patient is useful in instances where the number of viral particles is below the detection limits of current techniques. The detection of viral nucleic acid can be accomplished by, for example, (a) removing a sample to be tested from a patient; (b) treating the sample to release the viral DNA or RNA; (c) contacting the sample with a luminescent conjugate comprising a quantum dot material and a targeting moiety as described above, wherein the targeting moiety binds to the nucleic acid of the virus; and (d) detecting the luminescence, wherein the detection of luminescence indicates that the virus is present in the sample.

### Diagnostic Compositions

In another aspect, the present disclosure provides compositions comprising one or more of quantum dots as described above and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

Diagnostic compositions comprising the quantum dots(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used diagnostically.

Diagnostic compositions may take a form suitable for virtually any mode of administration, including, for example, topical, ocular, oral, buccal, systemic, nasal, injection, transdermal, rectal, vaginal, etc.

Useful injectable preparations include sterile suspensions, solutions or emulsions in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the quantum dot(s) may be dried by any art-known technique, such as lyophilization, and reconstituted prior to use.

For oral administration, the preparations may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For prolonged delivery, the quantum dots(s) can be formulated as a depot preparation for administration by implantation or intramuscular injection. The quantum dots(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, other diagnostic delivery systems may be employed. Liposomes and emulsions are well-known examples of delivery vehicles that may be used to deliver quantum dots(s). Certain organic solvents such as dimethylsulfoxide (DMSO) may also be employed, although usually at the cost of greater toxicity.

The diagnostic compositions may, if desired, be presented in a pack or dispenser device which may contain one or more unit dosage forms containing the quantum dots(s). The pack may, for example, comprise metal or plastic foil, such as a blister pack. The pack or dispenser device may be accompanied by instructions for administration.

The quantum dots(s) described herein, or compositions thereof, will generally be used in an amount effective to achieve the intended result. The amount of quantum dots(s) administered will depend upon a variety of factors, including, for example, the particular application for which the quantum dot is used, the mode of administration, whether the desired benefit is prophylactic or therapeutic, the age and weight of the patient, the bioavailability of the particular quantum dots(s) under the selected route of administration, etc.

Determination of an effective dosage of quantum dots(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration. Calculating dosages to achieve such circulating blood or serum concentrations taking into account the bioavailability of the composition via the desired route of administration is well within the capabilities of skilled artisans. Initial dosages can also be estimated from in vivo data, such as animal models. Animal models useful for testing the efficacy of the various compositions are well-known in the art. Animal models suitable for testing the bioavailability and/or metabolism of compounds into active metabolites are also well-known. Ordinarily skilled artisans can routinely adapt such information to determine dosages of particular compounds suitable for human administration.

### Methods of Preparation

The quantum dots of the present disclosure may be prepared by use of known chemical reactions and procedures. Those having skill in the art will recognize that the starting materials and reaction conditions may be varied, the sequence of the reactions altered, and additional steps employed to produce quantum dots encompassed by the present disclosure, as demonstrated by the following examples. Many general references providing commonly known chemical synthetic schemes and conditions useful for synthesizing the disclosed compounds are available.

Starting materials can be obtained from commercial sources or prepared by well-established literature methods known to those of ordinary skill in the art. The reactions are performed in a solvent appropriate to the reagents and materials employed and suitable for the transformations being effected. It will be understood by those skilled in the art of chemical synthesis that the functionality present on the molecule should be consistent with the transformations proposed. This will sometimes require a judgment to modify the order of the synthetic steps or to select one particular process scheme over another in order to obtain a desired compound of the disclosure.

### EXAMPLES

The preparation of the quantum dots(s) of the disclosure is illustrated further by the following examples, which are not to be construed as limiting the disclosure in scope or spirit to the specific procedures and quantum dots(s) described in them. Unless specified, all chemicals used were of analytical grade. All solutions were prepared in Milli-Q water (Millipore).

### Example 1

### Synthesis of Zn₁₋ₓAgₓSe

Zn₁₋ₓAgₓSe quantum dots 5-10 nm in average diameter were synthesized by swiftly injecting a freshly prepared NaHSe solution (0.4 M) into precursor solutions made up of zinc acetate (1 M, ZnAc₂), silver acetate (0.01 M, AgAc) and L-glutathione (GSH). The pH values of the reactions were adjusted with 10 M NaOH. The molar ratio of ZnAc₂:Se:GSH used in the experiments was kept at 8:1-2.2:9.7-34 while ZnAc₂:AgAc was at 30-100:1. The growth temperature was set at 22° C. and the final size of the quantum dots was controlled by varying the growth time and pH. After the reaction, 2-propanol was added to the quantum dots until turbidity occurred; the nanocrystals were isolated by centrifugation and dried under vacuum. Table 1 shows the feed molar ratio of GSH-ZnSeAg QDs prepared according to the procedure above:

TABLE 1

| | Zn | Se | Ag | GSH | | Em |
|---|---|---|---|---|---|---|
| Sample | [mmol] | [mmol] | [mmol] | [mmol] | pH | [nm] |
| QD510* | 5.00 | 0.62 | 0.05 | 6.09 | 8 | 510 |
| QD620 | 3.00 | 0.82 | 0.10 | 12.61 | 5 | 620 |
| QD710 | 5.00 | 0.62 | 0.05 | 6.09 | 7 | 710 |

*Sample was dissolved in water and the initial emission was 720 nm. The sample was stored at room temperature for 1 month and the emission was found to have shifted to 510 nm.

### Example 2

### Absorption and Fluorescence Measurements

Absorption and fluorescence spectra were recorded at room temperature on a UV-3600 spectrophotometer (Shimadzu) and a fluorescence spectrophotometer (Cary Eclipse). At regular time intervals, aliquots of the solution were withdrawn for UV absorption and fluorescence characterization. The fluorescence quantum yield measurements were determined from the integrated fluorescence intensities of the quantum dots and Atto488 dye in borate buffer (M, QY=80% at 501 nm excitation) [Atto465, M, QY=55% at 453 excitation] was used as the reference. The quantum dots samples were diluted with 50 mM borate buffer (PH 8.3) to yield an absorbance of 0.1 at the excitation wavelength.

### Example 3

### Size and Structural Characterization

TEM images were obtained with a FEI Tecnai G² TwinTEM (200 kV). X-ray diffraction analysis of the vacuum dried powders were performed on a Philips X'Pert PRO MRD HR X-Ray Diffraction System with CuK α radiation (A=1.5405 Å).

Elemental analysis was performed using energy-dispersive X-ray spectroscopy (EDX) was also used to evaluate the quantum dot materials of the disclosure. Table 2 provides elemental composition for the quantum dot of the disclosure, QD710, obtained from EDX experiments:

TABLE 2

| Mole Ratio | Zn | Se | S | Ag |
|---|---|---|---|---|
| Feed Ratio | 8.06 | 1.00 | 9.82 | 0.08 |
| Observed Ratio | 2.27 | 1 | 2.39 | 0.03 |

### Example 4

### Preparation of Heparin Conjugates

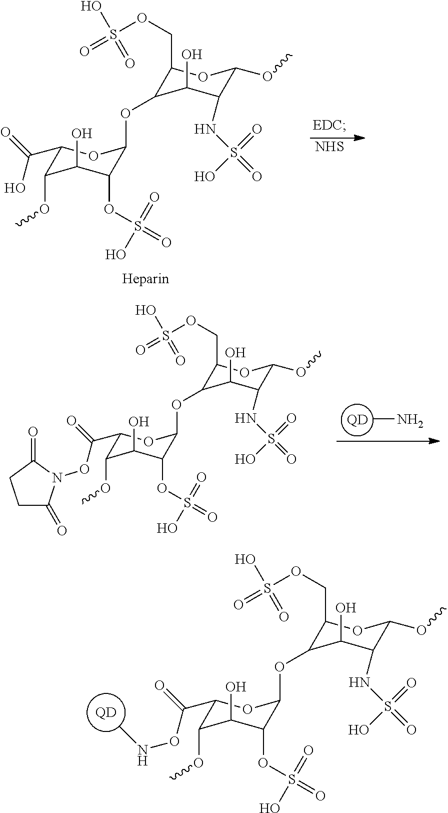

The conjugation was carried out in 0.1 M MES buffer at pH 6. Briefly, 4.5 mL of heparin (1 mg/mL), 4.3 mg of EDC (1-Ethyl-3-[3-dimethylaminopropyl]carbodiimide hydrochloride) and 6.1 mg NHS (N-hydroxysulfosuccinimide) were allowed to react at room temperature. After 30 min, 5 mL of quantum dot solution (10 mg/mL) was added and the reaction was allowed to continue for 4 h. The reaction was quenched with 6.3 mg of hydroxylamine hydrochloride and the heparin-conjugated quantum dot material was purified by ultrafiltration with 50K MWCO membrane.

### Example 5

### Preparation of Chitosan/Heparin Nanocomplexes

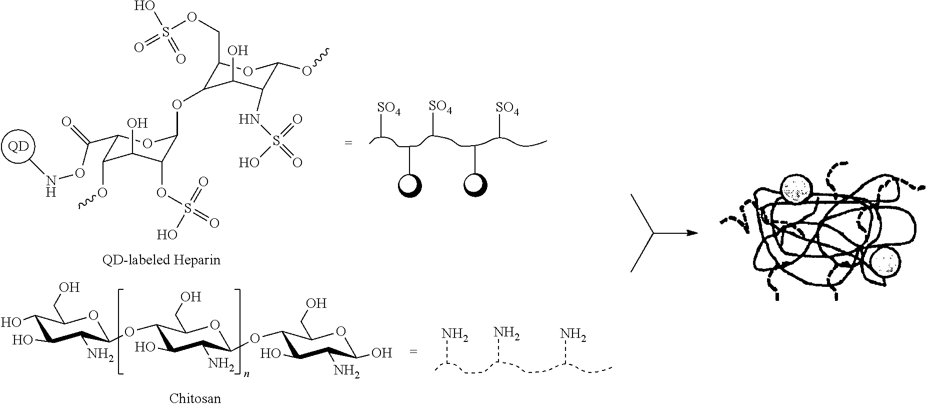

Chitosan (0.1 wt %, 0.2 mL) in sodium acetate buffer (pH 5.2, 0.2 M) was added to 1 mL of heparin-QDs dissolved in water (1 wt %) and mixed by vortex for 1 min to form the nanocomplexes. The process was repeated another four times until the volume of chitosan to heparin-QDs was about 1:1. The nanocomplexes were allowed to stand at 4° C. for 30 min, and the top layer was removed and replaced with PBS.

### Example 6

### In Vivo Biodistribution Study

Nude mice (Nu/Nu, 6-8 weeks old) were purchased from Charles River Laboratories and placed under purified diet (Harlan Laboratories, Adjusted Vitamins Diet TD.94096) one week prior and throughout the experiments. For injection and imaging, the mice were anaesthesized by inhalation of 2% isofluorane with oxygen. Quantum dots in phosphate buffered saline (N=3 mice each) were subcutaneously administered in the rear footpad at a dose of 50 mg/mL for QD710 and 0.16 μM for QD800-NH₂ respectively (50 μL each) while control mice were injected with 50 μL of PBS. The mice were imaged immediately post-injection with 675 nm excitation and 820 nm emission filter using the IVIS Spectra imaging system. The mice were sacrificed 1 day, 3 days and 7 days after injection, and the liver, intestines, stomach, kidney, spleen and popiteal lymph nodes were collected and imaged.

### Example 7

### In Vivo Trafficking Study of Chitosan/Heparin Nanocomplexes

Nude mice (Nu/Nu, 6-8 weeks old) were purchased from Charles River Laboratories and placed under purified diet (Harlan Laboratories, Adjusted Vitamins Diet TD.94096) one week prior and throughout the experiments. For injection and imaging, the mice were anaesthesized by inhalation of 2% isofluorane with oxygen. 50 μL of the nanocomplexes in phosphate buffered saline (N=3 mice each) were subcutaneously administered in the rear footpad at a dose while control mice were injected with 50 μL of PBS. The mice were imaged immediately and 4 hours post-injection with 675 nm excitation and 820 nm emission filter using the IVIS Spectra imaging system.

### Results

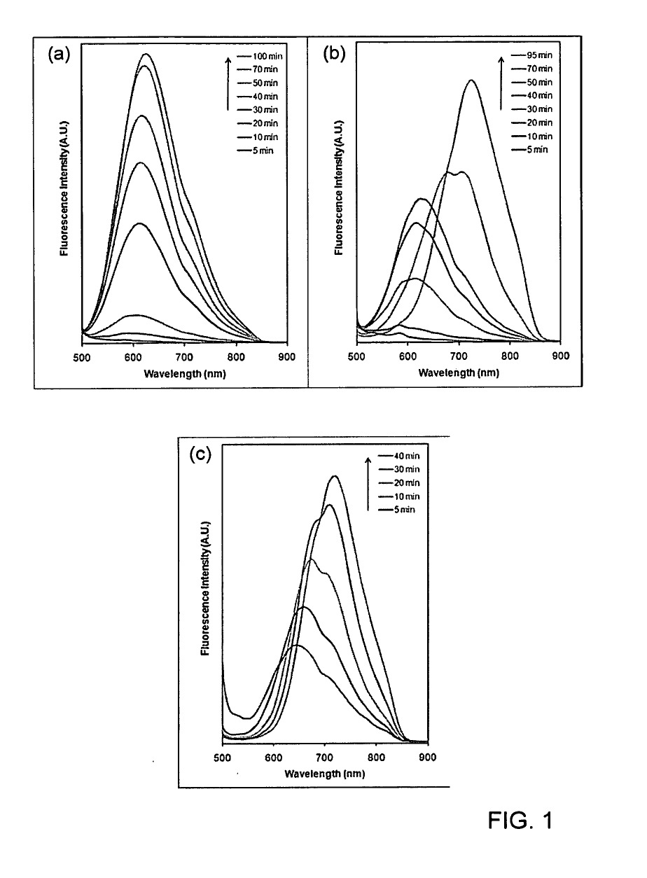

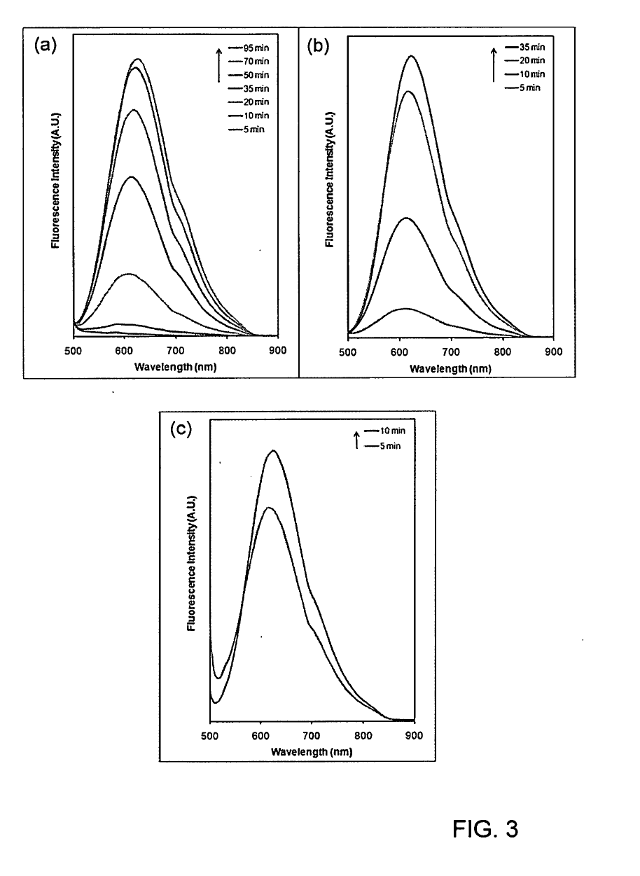

The NaHSe formed is subsequently added to a reaction flask containing a mixture of zinc acetate (ZnAc₂) and silver acetate (AgAc) with glutathione (GSH) as stabilizer. A change in the color of the solution ranging from yellow to red is observed. It was realized that reaction rate can be tuned by altering the pH of the reaction and amount of silver acetate used, while the size of the quantum dots can be varied by the concentration of GSH. FIG. 1 shows that as the pH of the reaction is increased, that rate of reaction increases. In addition, the rate of reaction increases with concentration of Ag⁺ as depicted in FIG. 3. The presence of Ag⁺ might have two effects, as a dopant in the quantum dots and secondly to catalyze the reaction.

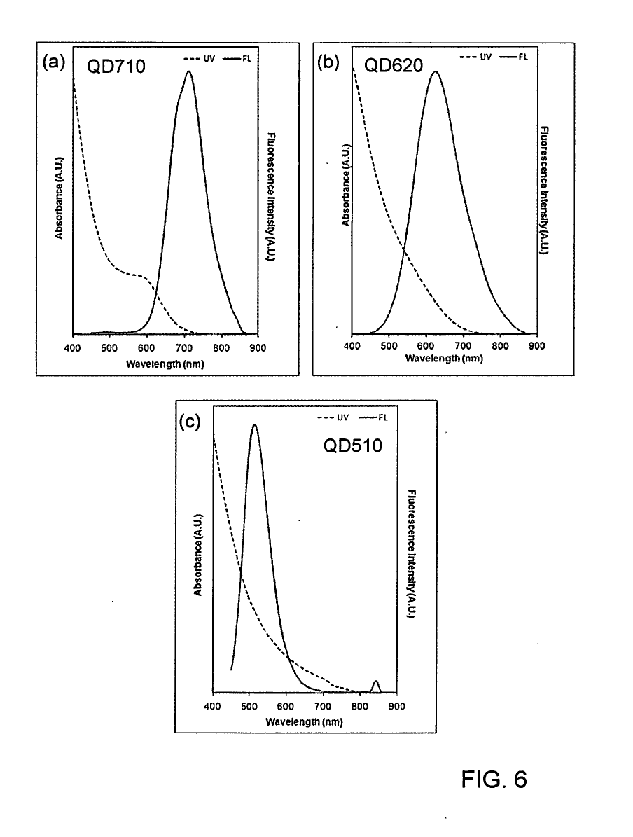

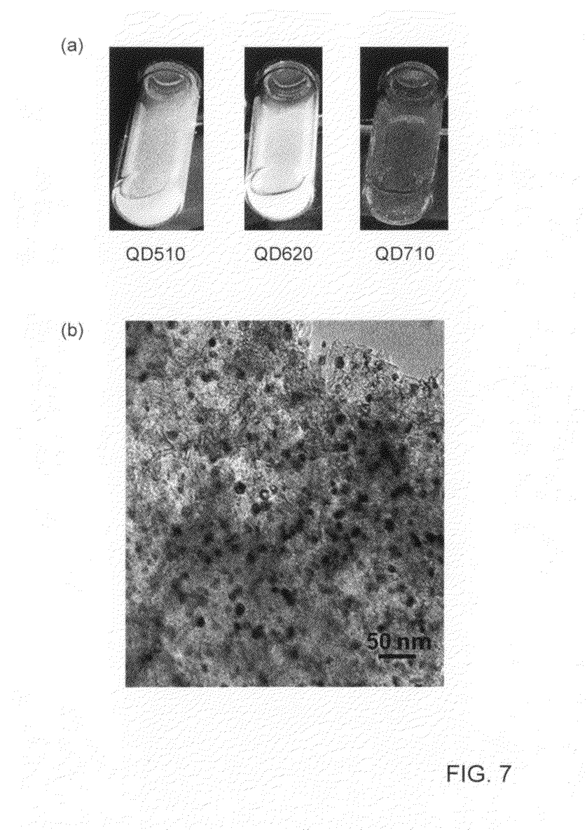

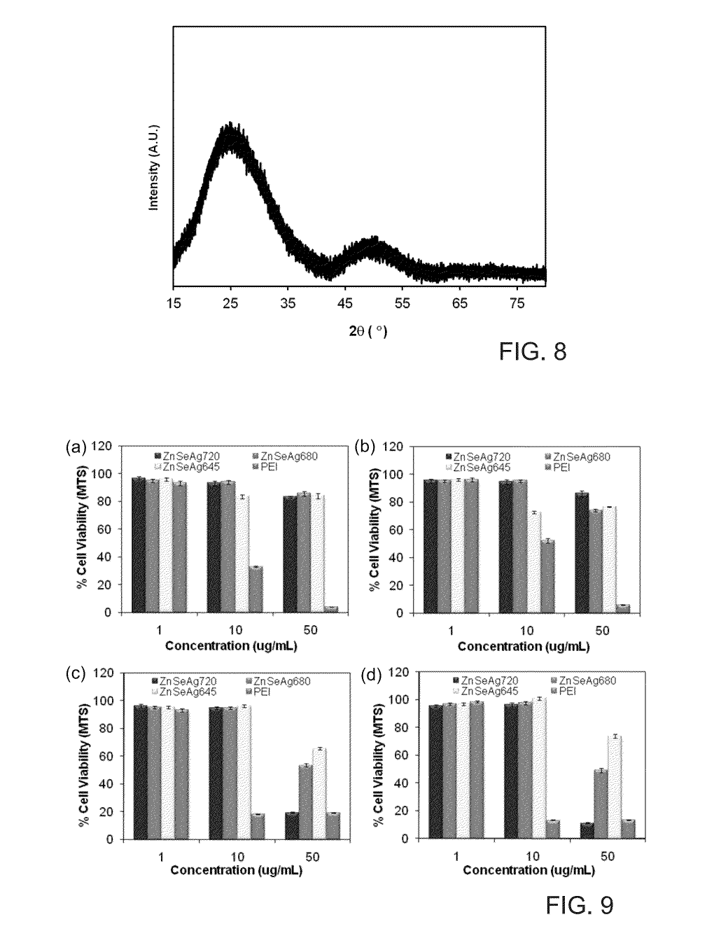

UV absorption and fluorescence spectra of samples with emission of 510, 620 and 710 nm are illustrated in FIG. 6, and FIG. 7(a) is a photograph of their emission. TEM images of indicated the average nanoparticle (NP) sizes vary from ˜2-10 nm (7(b)), corresponds to what is generally observed for quantum dots system that as the NP size increases, the emission would experience a blue shift. We further investigated the crystal structure of the sample with emission 710 nm (FIG. 8), found that it has a zinc blende cubic crystal structure and the estimated NP size based on the equation (Eq.) is ˜0.71 nm.

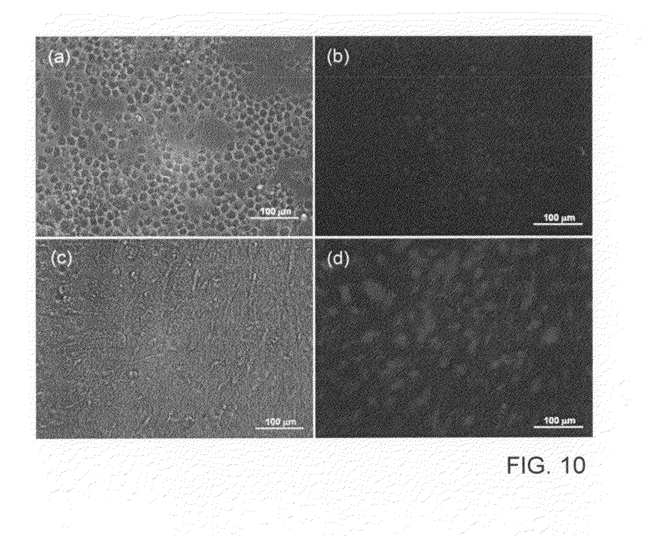

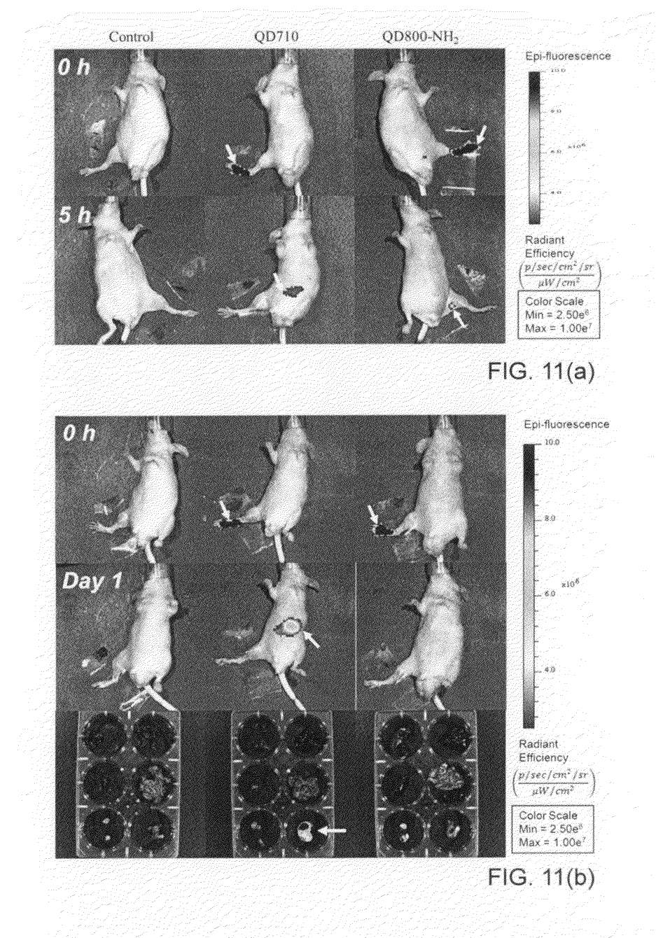

The cytotoxicity of the Zn₁₋ₓAgₓSe quantum dots was evaluated in four different cell lines, RAW 264.7 cells, hMSC, HUVEC, and HepG2 cells. As shown in FIG. 9a-b, RAW 264.7 cells maintained at least above 75% viability treated with the quantum dots for 24 hand 48 h. hMSC cells treated with 50 μg/mL of quantum dots for duration of 24 hand 48 h showed a drop in viability for cells (FIG. 9c-d). More quantum dots could enter into the cells and thus give rise to the toxicity as shown in FIGS. 10b and 10d where hMSC treated with QD710 produced higher fluorescence signals.

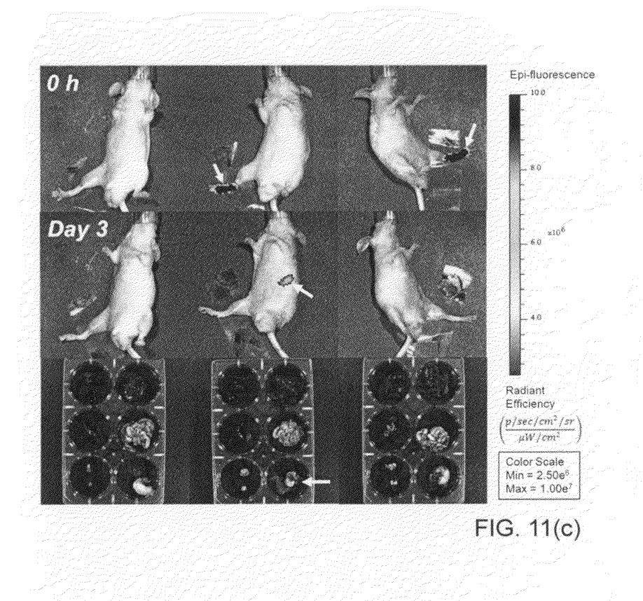

Zn₁₋ₓAgₓSe quantum dots were tracked in Nu/Nu mice after footpad injections. Biodistribution of QD710 and commercially available Qdot 800 ITK amino (PEG) quantum dots (Invitrogen) (QD800-NH₂) was evaluated (FIGS. 11(a)-(d)). Fluorescence signals were observable up to 7 days post treatment for both type of QDs. QD800-NH₂ QDs traversed through the lymphatic vessels and accumulated at the popiteal lymph nodes (LN) 5 h-7 days post treatment. In contrast, QD710 QDs were circulated through systemic route post-injection and accumulated in the stomach for as long as 7 days.

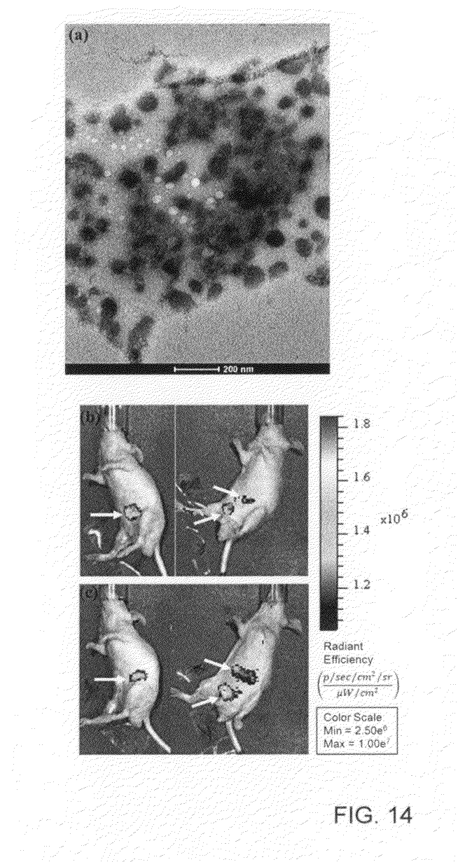

Similarly, chitosan/heparin nanocomplexes (NCs) were evaluated for their biodistribution, FIG. 14(a) shows the chitosan/heparin NCs with an average diameter ˜60-120 nm. The mice were imaged 0 and 4 hours post injection after chitosan/heparin NCs treatment (right panels). Chitosan/heparin NCs were shown to exhibit similar observations as bare QD710, and accumulated in the stomach of the mice (FIGS. 14 (b) and (c).)

It is to be understood that the examples and embodiments described herein are for illustrative purposes only. Unless clearly excluded by the context, all embodiments disclosed for one aspect of the invention can be combined with embodiments disclosed for other aspects of the invention, in any suitable combination. It will be apparent to those skilled in the art that various modifications and variations can be made to the present invention without departing from the scope of the invention. Thus, it is intended that the present invention cover the modifications and variations of this invention provided they come within the scope of the appended claims and their equivalents.

What is claimed is:

1. A quantum dot material comprising a plurality of particles, each particle comprising
a core having the formula Zn₁₋ₓAgₓY in which Y is S, Se or Te, and x is 0.001-0.05; and
at least a partial layer of stabilizing ligand substantially surrounding the core, the stabilizing ligand being glutathione, thioglycolic acid, 3-mercaptopropionic acid, mercaptoethanol, 3-mercaptopropane-1,2-diol, thiolactic acid, cysteine, cysteamine, or a combination thereof;
wherein the particles have an average diameter in the range of about 2 nm to about 50 nm.

2. A quantum dot material according to claim 1, wherein Y is Se or Te.

3. A quantum dot material according to claim 1, wherein Y is Se.

4. A quantum dot material according to claim 1, wherein Y is Te.

5. A quantum dot according to claim 1, wherein x is 0.001-0.03.

6. A quantum dot material according to claim 5, wherein x is 0.01-0.03.

7. A quantum dot material according to claim 1, where the stabilizing ligand is glutathione, thioglycolic acid, or 3-mercaptopropionic acid.

8. A quantum dot material according to claim 1, where the stabilizing ligand is glutathione.

9. A quantum dot material according to claim 1, where the material has a fluorescence emission at a wavelength in the visible or near-infrared.

10. A quantum dot material according to claim 9, wherein the wavelength is about 620 nm to about 900 nm.

11. A quantum dot material according to claim 1, wherein the particles have an average diameter in the range of about 2 to about 15 nm.

12. A diagnostic composition comprising a quantum dot material according to claim 1 and a diagnostically acceptable carrier, solvent, adjuvant or excipient.

13. A method of preparing a quantum dot material according to claim 1, the method comprising:
(a) mixing a Zn precursor, a Ag precursor, and the stabilizing ligand in a solvent;
(b) adding a solution containing a Y precursor to the mixture of (a);
(c) allowing the plurality of particles to form; and
(d) precipitating the formed quantum dot.

14. A method according to claim 13, wherein the stabilizing ligand is glutathione, thioglycolic acid, or 3-mercaptopropionic acid.

15. A method according to claim 13, wherein Y is Se or Te.

16. A method according to claim 13, wherein the molar ratio of Zn precursor to Y precursor is between 10:1 and 5:1.

17. A method according to claim 13, wherein the molar ratio of Zn precursor to the stabilizing ligand is between 1:1 and 1:4.

18. A method according to claim 13, wherein the solvent is water.

19. A method according to claim 13, performed at room temperature.

20. A method according to claim 13, wherein the time between performing step (b) and step (d) is 15 min to 120 min.

21. A conjugate comprising a quantum dot material according to claim 1 and a nanoparticle.

22. A conjugate comprising a quantum dot material according to claim 1 and a targeting moiety.

23. A method for monitoring delivery of a targeting moiety to an organism or biomaterial, the method comprising contacting the organism or biomaterial with a conjugate according to claim 22, and analyzing the cell to detect one or more of the presence, absence, amount and location of the conjugate in the organism or biomaterial.

24. A method for monitoring delivery of a targeting moiety to a subject comprising administering to a subject an effective amount of a conjugate according to claim 22, and detecting the presence, absence, or amount of the conjugate.

25. A method according to claim 22, wherein the detection is magnetic resonance imaging (MRI), X-ray imaging, computed tomography (CT), or electron spin resonance (ESR) imaging.

26. A method according to claim 25, wherein the detection is performed without the further addition of a contrast reagent.
